(19) United States
(12) Patent Application Publication
(10) Pub. No.: US 2026/0033771 A1
(43) Pub. Date: Feb. 5, 2026

(54) MODULAR BRAIN-COMPUTER INTERFACE WITH SCALABLE CHANNEL CAPACITY

(71) Applicant: NTL Group, Inc., Cave Creek, AZ (US)

(72) Inventors: Curtis Cripe, Cave Creek, AZ (US); Reagan Gillespie, Anthem, AZ (US)

(73) Assignee: NTL Group, Inc., Cave Creek, AZ (US)

(21) Appl. No.: 18/789,673

(22) Filed: Jul. 31, 2024

(51) Int. Cl. A61B 5/372

(52) U.S. Cl. CPC A61B 5/372

(57) ABSTRACT

Aspects of the disclosure are directed to Electroencephalography (EEG) staircase shifting. According to one aspect, the disclosure includes establishing a first control plane communication between a main module and a first of sub modules with source control lines; establishing a second control plane communication between the first and a second of sub modules with the source control lines using a staircase shifting; and activating at least one sub modules using the staircase shifting with the source control lines. In another aspect, the disclosure includes sub modules configured to acquire EEG signals; a main module coupled to the sub modules, the main module configured to serve as a data orchestrator for the sub modules, and transmission lines coupled to the main module and the submodules, wherein the transmission lines is configured to transport control lines, and wherein a systematic assignment of the transmission lines is implemented without external interconnection modification.

### TECHNICAL FIELD

This disclosure relates generally to the field of Electroencephalography (EEG), and, in particular, to EEG scalable channel capacity.

### BACKGROUND

An EEG (Electroencephalography) system monitors spatially distributed electrical signals from the brain using an EEG instrument. The EEG instrument generates a plurality of EEG signals which undergo signal processing prior to data interpretation. As the quantity of EEG signals increases, a scalable and modular EEG system is needed.

### SUMMARY

The following presents a simplified summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In one aspect, the disclosure provides Electroencephalography (EEG) staircase shifting. Accordingly, the present disclosure discloses an apparatus including: a plurality of sub modules each configured to acquire an Electroencephalography (EEG) signal; a main module coupled to the plurality of sub modules, the main module configured to serve as a data orchestrator for the plurality of sub modules; and a plurality of transmission lines coupled to the main module and the plurality of submodules, wherein the plurality of transmission lines is configured to transport a plurality of control lines, and wherein a systematic assignment of the plurality of transmission lines is implemented without external interconnection modification.

In one example, the apparatus further includes an Electroencephalography (EEG) electrode coupled to one of the plurality of sub modules, the EEG electrode configured to receive the EEG signal from a brain. In one example, the apparatus further includes an Electroencephalography (EEG) amplifier coupled to the EEG electrode, the EEG amplifier configured to amplify the EEG signal. In one example, the apparatus further includes a filter coupled to the EEG amplifier, the filter configured to restrict the EEG signal to a bandwidth. In one example, the apparatus further includes an analog-to-digital converter (ADC) coupled to the filter, the ADC configured to convert the EEG signal to a digitized Electroencephalography (EEG) signal. In one example, the main module is further configured to assign one of a plurality of inputs of the plurality of control lines to one of a plurality of outputs of the plurality of control lines to implement a unique control line to each of the plurality of sub modules.

Another aspect of the disclosure provides a method including: establishing a first control plane communication between a main module and a first of a plurality of sub modules with a plurality of source control lines; establishing a second control plane communication between the first and a second of the plurality of sub modules with the plurality of source control lines using a staircase shifting; and activating at least one of the plurality of sub modules using the staircase shifting with the plurality of source control lines. In one example, the at least one of the plurality of sub modules includes the first of the plurality of sub modules or the second of the plurality of sub modules.

In one example, the method further includes designating one of the plurality of source control lines for communicating with one of the plurality of sub modules. In one example, the method further includes assigning one of a plurality of inputs of the plurality of source control lines to one of a plurality of outputs of the plurality of source control lines to implement a unique source control line to each of the plurality of sub modules. In one example, the method further includes acquiring and digitizing a plurality of Electroencephalography (EEG) channels from the at least one of the plurality of sub modules for a first Electroencephalography (EEG) data processing and analysis.

In one example, the method further includes establishing a data plane communication between the main module and the plurality of sub modules in an Electroencephalography (EEG) system with an outbound serial data interface and an inbound serial data interface. In one example, the method further includes activating one or more of the plurality of sub modules using the staircase shifting with the plurality of source control lines for a second Electroencephalography (EEG) data processing and analysis, wherein the one or more of the plurality of sub modules does not include any of the at least one of the plurality of sub modules.

Another aspect of the disclosure provides a non-transitory computer-readable medium storing computer executable code, operable on a device including at least one processor and at least one memory coupled to the at least one processor, wherein the at least one processor is configured to implement Electroencephalography (EEG) scalable channel capacity, the computer executable code including: instructions for causing a computer to establish a first control plane communication between a main module and a first of a plurality of sub modules with a plurality of source control lines; instructions for causing the computer to establish a second control plane communication between the first and a second of the plurality of sub modules with the plurality of source control lines using a staircase shifting; and instructions for causing the computer to activate at least one of the plurality of sub modules using the staircase shifting with the plurality of source control lines.

In one example, the at least one of the plurality of sub modules includes the first of the plurality of sub modules or the second of the plurality of sub modules. In one example, the non-transitory computer-readable medium further includes instructions for causing the computer to designate one of the plurality of source control lines for communicating with one of the plurality of sub modules. In one example, the non-transitory computer-readable medium further includes instructions for causing the computer to assign one of a plurality of inputs of the plurality of source control lines to one of a plurality of outputs of the plurality of source control lines to implement a unique source control line to each of the plurality of sub modules.

In one example, the non-transitory computer-readable medium further includes instructions for causing the computer to acquire and digitize a plurality of Electroencephalography (EEG) channels from the at least one of the plurality of sub modules for a first Electroencephalography (EEG) data processing and analysis. In one example, the non-transitory computer-readable medium further includes instructions for causing the computer to establish a data plane communication between the main module and the plurality of sub modules in an Electroencephalography (EEG) system with an outbound serial data interface and an inbound serial data interface.

In one example, the non-transitory computer-readable medium further includes instructions for causing the computer to activate one or more of the plurality of sub modules using the staircase shifting with the plurality of source control lines for a second Electroencephalography (EEG) data processing and analysis, wherein the one or more of the plurality of sub modules does not include any of the at least one of the plurality of sub modules.

These and other aspects of the present disclosure will become more fully understood upon a review of the detailed description, which follows. Other aspects, features, and implementations of the present disclosure will become apparent to those of ordinary skill in the art, upon reviewing the following description of specific, exemplary implementations of the present invention in conjunction with the accompanying figures. While features of the present invention may be discussed relative to certain implementations and figures below, all implementations of the present invention can include one or more of the advantageous features discussed herein. In other words, while one or more implementations may be discussed as having certain advantageous features, one or more of such features may also be used in accordance with the various implementations of the invention discussed herein. In similar fashion, while exemplary implementations may be discussed below as device, system, or method implementations it should be understood that such exemplary implementations can be implemented in various devices, systems, and methods.

### BRIEF DESCRIPTION OF THE DRAWINGS

FIG. 1 illustrates an example Electroencephalography (EEG) channel signal acquisition equipment in an Electroencephalography (EEG) system.

FIG. 2 illustrates an example Electroencephalography (EEG) system sub module selection architecture.

FIG. 3 illustrates an example sub module selection interconnection with three sub modules.

FIG. 4 illustrates an example flow diagram for an Electroencephalography (EEG) system staircase shifting.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

While for purposes of simplicity of explanation, the methodologies are shown and described as a series of acts, it is to be understood and appreciated that the methodologies are not limited by the order of acts, as some acts may, in accordance with one or more aspects, occur in different orders and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with one or more aspects.

An EEG (electroencephalography) system monitors electrical signals from the brain for neurological disorder diagnosis. The EEG system may acquire a large quantity of spatially distributed electrical signals from the brain to generate a plurality of EEG signals. The plurality of EEG signals may be analyzed over a temporal dimension (i.e., time), over a spectral dimension (i.e., frequency) and over a spatial dimension (i.e., location) to assist in clinical diagnosis. Each EEG signal of the plurality of EEG signals may be a voltage waveform as a function of time. For example, each EEG signal may be analyzed in a spectral domain (e.g., via Fourier decomposition or via bandpass filtering) to generate a plurality of spectral band data for each EEG signal. The plurality of EEG signals may be received and digitized by EEG channel signal acquisition equipment prior to data reduction and analysis.

Current clinical applications may drive a need for a greater quantity of EEG signals to provide improved sampling over the spatial dimension. The higher quantity of EEG signals requires a scalable and modular EEG system to transform each EEG signal into a digitized EEG replica for subsequent processing and clinical diagnosis. The scalable and modular EEG system may facilitate an economic scaling of the EEG system over a wide range of EEG signal quantities. In one example, each EEG signal is acquired by a dedicated EEG channel in the EEG system. That is, the quantity of EEG signals equals the quantity of EEG channels in the EEG system. Each EEG channel may include signal acquisition hardware including an analog to digital converter (ADC) to generate the digitized EEG replica signal from each EEG signal. The digitized EEG replica forms the basis for EEG data (i.e., all digital data acquired and processed by the EEG system) for subsequent data reduction and analysis.

In one example, an EEG system may be limited to a finite EEG channel capacity (i.e., a maximum quantity of EEG signals). In one example, the EEG system may have limited flexibility in adapting to diverse user needs (e.g., applications ranging from consumer-grade EEG instrumentation to professional laboratory EEG research). In one aspect, there is a need for a scalable and modular EEG system which acquires high quality EEG data and is tailorable for diverse user requirements.

In one example, an EEG system may be implemented with a plurality of modules. A module is a functional unit with electronic circuitry for data transport and control with connectivity to a databus for inter-module communication. In one example, the plurality of modules may include one main module and a plurality of sub modules. In one example, the main module (e.g., master module or controller) serves as a data orchestrator for the plurality of sub modules (e.g., peripheral modules). In one example, for a scalable and modular EEG system, the plurality of sub modules may be physically identical, that is, with the same mechanical dimensions, electrical layout and interconnection locations. In one example, physically identical sub modules may facilitate an economic scaling of the EEG system over a wide range of EEG signal quantities and a tailorable architecture for diverse user requirements.

In one example, the EEG system includes a plurality of EEG channels where each EEG channel includes EEG signal acquisition equipment. For example, the plurality of EEG channels may be divided into groups of EEG channels. For example, if the plurality of EEG channels has 64 EEG channels, the plurality of EEG channels may be divided into 8 groups of 8 EEG channels each or into 4 groups of 16 EEG channels each. In one example, each group of EEG channels may be assigned to one sub module. That is, each sub module may acquire and process one group of EEG channels. In one aspect, the quantity of EEG channels and/or the quantity of groups of EEG channels may depend on a particular application and/or design choice. One skilled in the art would understand that the quantity of EEG channels and/or the quantity of groups of EEG channels may vary from the disclosed examples and still be within the scope and spirit of the present disclosure.

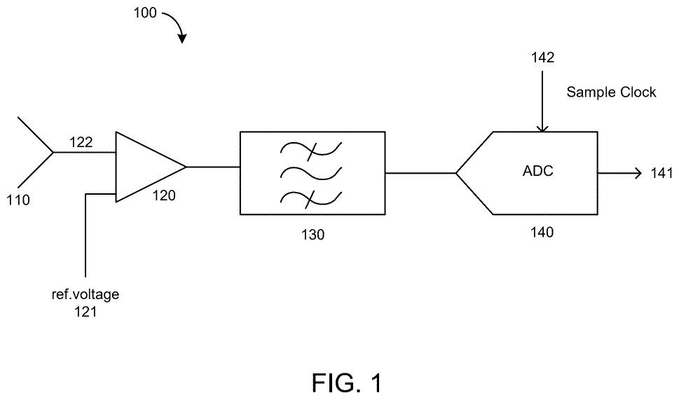

FIG. 1 illustrates an example Electroencephalography (EEG) channel signal acquisition equipment 100 in an Electroencephalography (EEG) system. In one example, the EEG system includes a main module and a plurality of sub modules. In one example, the EEG channel signal acquisition equipment 100 is within a sub module of the EEG system. In one example, the EEG channel signal acquisition equipment 100 acquires and processes one EEG channel of a group of EEG channels. In one example, each sub module of a plurality of sub modules of an EEG system acquires and processes one group of EEG channels. In one example, a plurality of sub modules of the EEG system acquires and processes a plurality of groups of EEG channels (i.e., a plurality of EEG channels).

In one example, the EEG channel signal acquisition equipment 100 includes an EEG electrode 110, an EEG signal amplifier 120, a filter 130 and an analog-to-digital converter (ADC) 140. In one example, the EEG electrode 110 receives an electrical signal 122 from a region of a brain (e.g., human brain) near the placement of the EEG electrode 110 and sends the electrical signal 122 to the EEG signal amplifier 120 for amplification, to the filter 130 for restricting the electrical signal to a signal bandwidth B and to the ADC 140 for conversion to a digitized EEG signal 141. In one example, the digitized EEG signal 141 is an output of the EEG channel signal acquisition equipment 100 and is sent to a digital signal processor and computer.

In one example, the EEG signal amplifier 120 is a differential amplifier with a reference voltage 121 as a secondary input. In one example, the secondary input may be another EEG electrode. In one example, the ADC 140 samples its input with a sample clock 142 with a sampling frequency consistent with the signal bandwidth B established by the filter 130. For example, the sampling frequency may be at least a factor of two greater than the signal bandwidth B.

In one example, a sub module of the EEG system includes a fixed quantity of EEG channels. In one example, the fixed quantity of EEG channels is 8 or a multiple of 8. In one example, the EEG system is scalable by replicating a sub module with the fixed quantity of EEG channels. In one example, the EEG system may be a dual stack design where a stack is an electrically connected plurality of modules using a single databus for data connectivity. One skilled in the art would understand that the fixed quantity disclosed herein is an example, and other quantities are within the scope and spirit of the present disclosure.

For example, the EEG system (e.g., having identical sub module design) may be used for adaptability and future expansion. In one example, the EEG system may include an integrated bias level and voltage reference sharing among all modules for signal integrity maintenance. In one example, the EEG system may utilize a common synchronous clock for time-aligned (i.e., synchronous) and frequency-aligned (i.e., syntonous) data registration. In one example, each sub module of the EEG system may be individually addressable for on-the-fly adjustment of equipment settings.

In one example, the EEG system may include internal dc power line filtering for stable data transport operation (e.g., using wired data transport, such as USB or Ethernet). In one example, the EEG system may include wireless data transport (e.g., Bluetooth low energy (BLE)) for untethered applications. In one example, the EEG system may be compatible with data analysis software (e.g., Brainflow). In one example, the EEG system may include an optional back clip for versatile equipment placement options.

In one example, the EEG system processes data plane traffic and control plane traffic. In one example, data plane traffic includes information directly derived from the plurality of EEG signals. In one example, control plane traffic includes information for configuration of the EEG system and routing of the EEG signal and EEG data. For example, control plane traffic is not directly derived from the plurality of EEG signals.

In one example, the EEG system may use a serial peripheral interface (SPI) databus to interconnect the main module and the plurality of sub modules for both data plane traffic and control plane traffic. In one example, the SPI databus adheres to a communications protocol which includes two shared data lines for data transport (e.g., an outbound serial data interface, an inbound serial data interface), one shared clock line (e.g., a common synchronous clock signal) and a dedicated control line for control plane traffic per sub module. In one example, the dedicated control line allows the main module to designate which sub module it is communicating with.

For example, the dedicated control line is activated for a specific sub module to avoid any collisions with other sub modules. In one example, outbound refers to a direction from the main module to the plurality of sub modules. In one example, inbound refers to a direction from the plurality of sub modules to the main module. In one example, the dedicated control line is transported (i.e., carried) by a transmission line. In one example, a plurality of transmission lines interconnect the main module and the plurality of sub modules. In one example a systematic assignment of the plurality of transmission lines is implemented without external interconnection modification. In one example, a plurality of control lines is used to convey control plane traffic such as configuration information.

In one example, the SPI databus may include an outbound serial data interface to relay outbound data plane traffic from the main module to the plurality of sub modules. For example, the SPI databus may include an inbound serial data interface to relay inbound data plane traffic from the plurality of sub modules to the main module. For example, the SPI databus may also include a common synchronous clock signal which originates from the main module and is distributed to the plurality of sub modules as a plurality of replicas of the common synchronous clock signal. In one example, synchronous refers to a relative timing difference which is less than a specified fraction of a clock period among the plurality of replicas of the common synchronous clock signal.

In one example, the SPI also includes a chip select (CS) binary signal which enables or disables data transport between the main module and one sub module out of the plurality of sub modules. In one example, the CS binary signal may be an active high signal (i.e., a high voltage indicates enable and a low voltage indicates disable). In one example, the CS binary signal may be an active low signal (i.e., a low voltage indicates enable and a high voltage indicates disable). In one example, no more than one sub module may be enabled for data transport at a time. In one example, the data transport may be simplex (i.e., unidirectional) or duplex (i.e., bidirectional) between the main module and an enabled sub module. In one example, one bit or one data symbol is transport per synchronous clock cycle.

In one example, there is one CS binary signal for each sub module. For example, if there are N sub modules in the EEG system, there are N CS binary signals, where N is a positive integer. In one example, CS1 denotes a CS binary signal for sub module 1, CS2 denotes a CS binary signal for sub module 2, and so on until CSN denotes a CS binary signal for sub module N.

In one example, the main module selects one sub module for data transport by enabling its corresponding CS binary signal. For example, if sub module k is selected for data transport, then CSk is enabled, where k is a positive integer ranging from k=1 to k=n.

In one example, an EEG system includes a physical design where each sub module is packaged as a stackable module for ease of modular expansion. In one example, each stackable module is physically identical with other stackable modules, that is, with the same mechanical dimensions, electrical layout and interconnection locations. In one example, each stackable module is physically connected to another stackable module with electrical mating from a transmitting stack header to a receiving stack header. In one example, each stackable module (i.e., each sub module) should be activated independently while using an identical interconnection architecture without a software addressing scheme. One skilled in the art would understand that the present disclosure does not exclude another example wherein a stackable module may have different mechanical dimensions, electrical layout and/or interconnection locations with another stackable module. And, in one example, differences between stackable modules may be based on particular applications.

In one example, staircase shifting implements a systematic reassignment of control line (e.g., chip select (CS) line) assignments without external interconnection modifications. In one example, staircase shifting redirects (e.g., shaves) one or more control lines (e.g., chip select (CS) lines) for each sub module to implement a unique control line (e.g., CS line) per sub module. In one example, other control lines (e.g., CS lines) are shifted at least one position, consistent with the quantity of redirected control lines (e.g., CS lines) per sub module. The process of redirection and shifting is repeated a quantity of n times which allows up to n sub modules to be added to the EEG system, where n is a positive integer. In one example, each sub module is independently addressable by the main module (e.g., stack controller).

In one example, staircase shifting allows physically identical sub modules to facilitate modular scalability of the EEG system. For example, modular scalability promotes economies of scale in manufacturing and seamless repair. In one example, staircase shifting allows agnostic positioning of sub modules in the EEG system and simplifies addressability by the main module since the CS lines are distributed to the plurality of sub modules sequentially. In one example, staircase shifting requires no customization or firmware flashing to prevent address collisions. In one example, any combination of n sub modules may be used as long as n CS lines are available from the main module, where n is a positive integer. In one example, a finite sequence of sub modules may be interconnected with one databus with a simple and high-speed architecture.

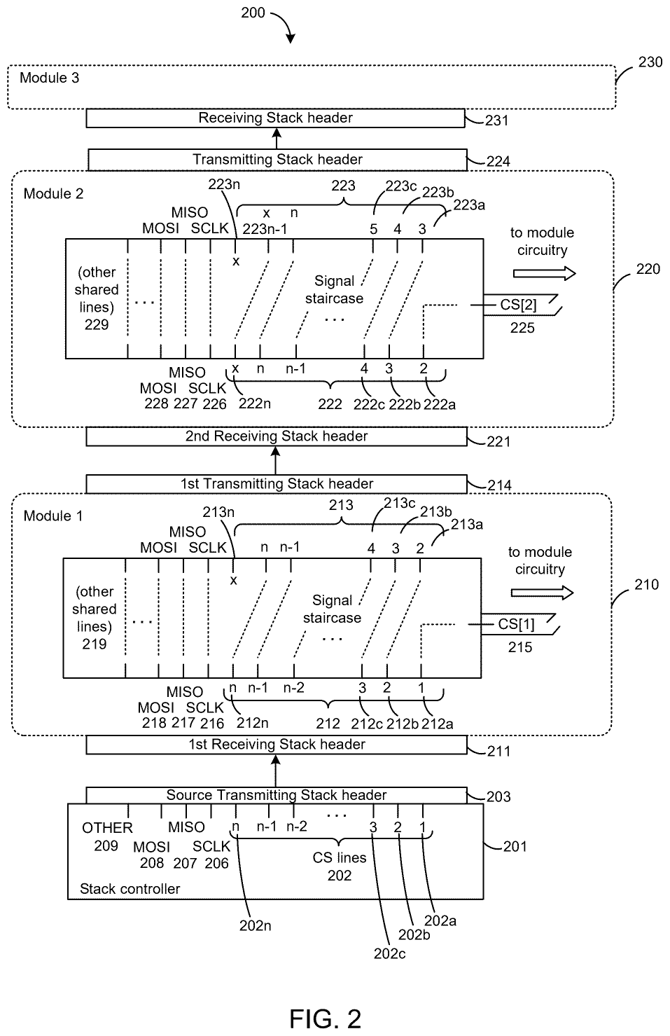

In one example, FIG. 2 illustrates an example Electroencephalography (EEG) system sub module selection architecture 200. In one example, the architecture 200 includes a main module 201, a first sub module 210, a second sub module 220, a third sub module 230. Although only 3 sub modules are shown in FIG. 2, one skilled in the art would understand that FIG. 2 illustrates an example and the present disclosure does not exclude other quantities of sub modules. That is, other quantities of sub modules are also within the scope and spirit of the present disclosure. In one example, the main module 201 serves as a stack controller, that is, a controller of a stack of sub modules.

In one example, the main module 201 includes a plurality of source chip select (CS) lines 202. In one example, each CS line carries a CS binary signal to select one sub module for data transport. In one example, CS1 denotes a CS binary signal for sub module 1, CS2 denotes a CS binary signal for sub module 2, and so on until CSn denotes a CS binary signal for sub module n, where n is a positive integer. For example, if sub module 1 is selected for data transport, then CS1 is enabled. For example, if sub module k is selected for data transport, then CSk is enabled, where k is a positive integer ranging from k=1 to k=n. For example, the plurality of source CS lines 202 includes a plurality of n lines, where n is a positive integer. For example, the plurality of source CS lines 202 includes a first source CS line 202a, a second source CS line 202b, a third source CS line 202c, and so on until an nth source CS line 202n.

In one example, the main module 201 also includes a source synchronous clock signal SCLK 206, a source main out sub in (MOSI) signal 208, a source main in sub out (MISO) signal 207, and other source signals 209. In one example, the main module 201 sends the plurality of source CS input lines 202, the source SCLK 206, the source MOSI signal 208, the source MISO signal 207 and other source signals 209 from a source transmitting stack header 203 to a first receiving stack header 211 in the first sub module 210.

In one example, the first sub module 210 includes a plurality of first sub module chip select (CS) input lines 212. For example, the plurality of first sub module CS input lines 212 includes a plurality of n lines, where n is a positive integer. For example, the plurality of first sub module CS input lines 212 includes a first sub module first CS input line 212a, a first sub module second CS input line 212b, a first sub module third CS input line 212c, and so on until a first sub module nth CS input line 212n. In one example, the plurality of first sub module CS input lines 212 is staircase shifted by an integral offset to produce a plurality of first sub module CS output lines 213. In one example, the integral offset is one. For example, the plurality of first sub module CS output lines 213 includes a first sub module first CS output line 213a, a first sub module second CS output line 213b, a first sub module third CS output line 213c, and so on until a first sub module nth CS output line 213n.

In one example, staircase shifting by the integral offset implements a systematic reassignment of CS line assignments without external interconnection modifications. For example, if the integral offset is one, staircase shifting assigns the first sub module first CS input line 212a to a first sub module circuitry enable line CS[1], the first sub module second CS input line 212b to the first sub module first CS output line 213a, the first sub module third CS input line 212c to the first sub module second CS output line 213b, the first sub module fourth CS input line 212d to the first sub module third CS output line 213c, and so on until the first sub module nth CS input line 212n to a first sub module (n−1)th CS output line 213n−1. In one example, a first sub module nth CS output line 213n is unterminated (i.e., not connected to an output) wherein the nth CS output line is labeled as “x” in the first sub module 210 of FIG. 2.

In one example, the first sub module 210 also includes a first synchronous clock signal SCLK 216, a first main out sub in (MOSI) signal 218, a first main in sub out (MISO) signal 217, and a first of other signals 219. In one example, the first sub module 210 sends the plurality of first CS input lines 212, the first SCLK 216, the first MOSI signal 218, the first MISO signal 217 and the first other signals 219 from a first transmitting stack header 214 to a second receiving stack header 221 in the second sub module 220.

In one example, the second sub module 220 includes a plurality of second sub module chip select (CS) input lines 222. For example, the plurality of second sub module CS input lines 222 includes a plurality of n lines, where n is a positive integer. For example, the plurality of second sub module CS input lines 222 includes a first CS input line 222a, a second CS input line 222b, a third CS input line 222c, and so on until an nth CS input line 222n, wherein the nth CS input line is labeled as “x” in the second sub module 220 of FIG. 2.

In one example, staircase shifting by the integral offset implements a systematic reassignment of CS line assignments without external interconnection modifications. For example, if the integral offset is one, staircase shifting assigns the second sub module first CS input line 222a to a second sub module circuitry enable line CS[2], the second sub module second CS input line 222b to the second sub module first CS output line 223a, the second sub module third CS input line 222c to the second sub module second CS output line 223b, the second sub module fourth CS input line 222d to the second sub module third CS output line 223c, and so on until the second sub module nth CS input line 222n to a second sub module (n−1)th CS output line 223n−1. In one example, a second sub module nth CS output line 223n is unterminated (i.e., not connected to an output) wherein the (n−1)th CS output line and the nth CS output line are labeled as “x” in the second sub module 220 of FIG. 2.

In one example, the second sub module 220 also includes a second synchronous clock signal SCLK 226, a second main out sub in (MOSI) signal 228, a second main in sub out (MISO) signal 227, and a second of other signals 229. In one example, the second sub module 220 sends the plurality of second CS input lines 222, the second SCLK 226, the second MOSI signal 228, the second MISO signal 227 and the second other signals 219 from a second transmitting stack header 224 to a third receiving stack header 231 in the third sub module 230.

In one example, the third sub module 230 includes a plurality of third sub module chip select (CS) lines (not shown). In one example, the third sub module 230 and any subsequent sub modules include staircase shifting as described above for the first sub module 210 and the second sub module 220.

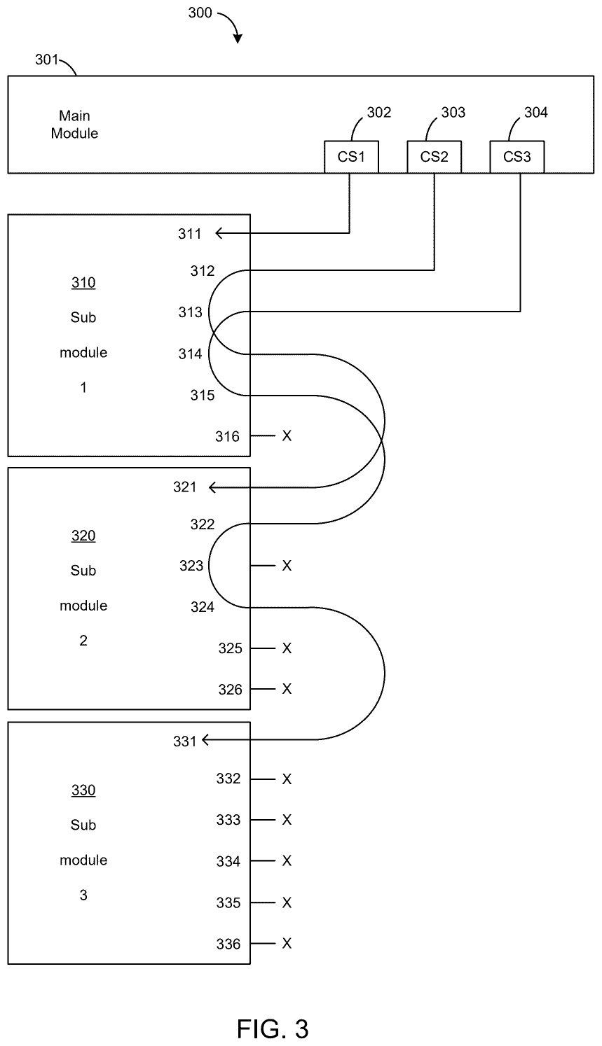

FIG. 3 illustrates an example sub module selection interconnection 300 with three sub modules. In one example, a main module 301 includes a first source CS output 302, a second source CS output 303 and a third source CS output 304. In one example, the three sub modules are physically identical.

In one example, a first sub module 310 includes a first sub module first CS input 311, a first sub module second CS input 312 and a first sub module third CS input 313. In one example, the first sub module 310 includes a first sub module first CS output 314, a first sub module second CS output 315 and a first sub module third CS output 316.

In one example, a second sub module 320 includes a second sub module first CS input 321, a second sub module second CS input 322 and a second sub module third CS input 323. In one example, the second sub module 320 includes a second sub module first CS output 324, a second sub module second CS output 325 and a second sub module third CS output 326.

In one example, a third sub module 330 includes a third sub module first CS input 331, a third sub module second CS input 332 and a third sub module third CS input 333. In one example, the third sub module 330 includes a third sub module first CS output 334, a third sub module second CS output 335 and a third sub module third CS output 336.

In one example, FIG. 3 illustrates an implementation of staircase shifting where the first source CS output 302 is connected to the first sub module first CS input 311, second source CS output 303 is connected to the first sub module second CS input 312, and the third source CS output 304 is connected to the first sub module third CS input 313.

Next, in one example, the first sub module first CS input 311 is connected to a first sub module circuitry enable line CS[1] (not shown), the first sub module second CS input 312 is connected to the first sub module first CS output 314 and the first sub module third CS input 313 is connected to the first sub module second CS output 315. In one example, the first sub module third CS output 316 is unterminated.

Next, in one example, the first sub module first CS output 314 is connected to the second sub module first CS input 321 and the first sub module second CS output 315 is connected to the second sub module second CS input 322. In one example, the second sub module third CS input 323 is unterminated.

Next, in one example, the second sub module first CS input 321 is connected to a second sub module circuitry enable line CS[2] (not shown), the second sub module second CS input 322 is connected to the second sub module first CS output 324. In one example, the second sub module second CS output 325 and the second sub module third CS output 326 are both unterminated.

Next, in one example, the second sub module first CS output 324 is connected to the third sub module first CS input 331. In one example, the second sub module second CS output 325 and the second sub module third CS output 326 are both unterminated.

Next, in one example, the third sub module first CS input 331 is connected to a third sub module circuitry enable line CS[3] (not shown). In one example, the third sub module second CS input 332, the third sub module third CS input 333, the third sub module first CS output 334, the third sub module second CS output 335 and the third sub module third CS output 336 are all unterminated.

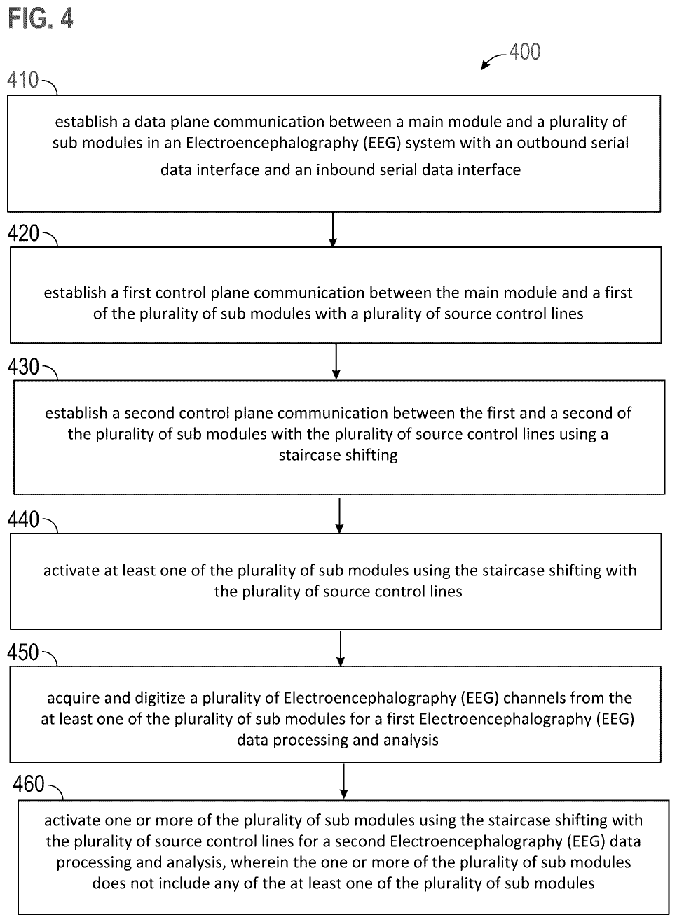

FIG. 4 illustrates an example flow diagram 400 for an Electroencephalography (EEG) system staircase shifting. In block 410, establish a data plane communication between a main module and a plurality of sub modules in an Electroencephalography (EEG) system with an outbound serial data interface and an inbound serial data interface. In one example, a data plane communication is established between a main module and a plurality of sub modules in an Electroencephalography (EEG) system with an outbound serial data interface and an inbound serial data interface.

In one example, the data plane communications is synchronized and syntonized with a common synchronous clock signal. In one example, the outbound serial data interface relays outbound data plane traffic from the main module to the plurality of sub modules. In one example, the inbound serial data interface relays inbound data plane traffic from the plurality of sub modules to the main module. In one example, the common synchronous clock signal originates from the main module and is distributed to the plurality of sub modules as a plurality of replicas of the common synchronous clock signal. In one example, synchronous refers to a relative timing difference which is less than a specified fraction of a clock period among the plurality of replicas of the common synchronous clock signal. In one example, the step of block 410 may be performed by one or more of the following: a main module, a stack controller, a microprocessor and/or a microcontroller.

In block 420, establish a first control plane communication between the main module and a first of the plurality of sub modules with a plurality of source control lines. In one example, a first control plane communication is established between the main module and a first of the plurality of sub modules with a plurality of source control lines.

In one example, the main module designates one of the plurality of source control lines for communicating with one of the plurality of sub modules. In one example, activating one designated control line for a specific sub module avoids collisions (e.g., traffic collisions) with other sub modules. In one example, the designating establishes a one-to-one relationship between the plurality of sub modules and the plurality of source control lines. In one example, the first control plane communication includes information for EEG system configuration. In one example, the first control plane communication includes information for routing of an EEG signal and EEG data. In one example, the step of block 420 may be performed by one or more of the following: a main module (e.g., main module 201 or main module 301), a stack controller, a microprocessor and/or a microcontroller.

In one example, the plurality of source control lines is used to convey configuration information of the plurality of sub modules by the main module. For example, configuration information may include signals for selection of data transport enablement. And, in one example, a plurality of source chip select (CS) lines is a plurality of source control lines.

In block 430, establish a second control plane communication between the first and a second of the plurality of sub modules with the plurality of source control lines using a staircase shifting. In one example, a second control plane communication is established between the first and a second of the plurality of sub modules with the plurality of source control lines using a staircase shifting.

In one example, each source control line includes an input and an output for each sub module. In one example, each sub module includes a plurality of source control lines. Staircase shifting assigns one of a plurality of inputs of the plurality of source control lines to one of a plurality of outputs of the plurality of source control lines to implement a unique source control line per sub module. In one example, each of the plurality of source control lines is shifted at least one position per sub module. In one example, each sub module is independently addressable by the main module using the plurality of source control lines. In one example, the step of block 430 may be performed by one or more of the following: a main module, a stack controller, a microprocessor and/or a microcontroller.

In block 440, activate at least one of the plurality of sub modules using the staircase shifting with the plurality of source control lines. In one example, at least one of the plurality of sub modules is activated using the staircase shifting with the plurality of source control lines. In one example, activation of the least one sub module uses staircase shifting implements a systematic reassignment of source control line assignments without external interconnection modifications. In one example, the step of block 440 may be performed by one or more of the following: a power manager (e.g., a device that manages dc power), a power controller, a microprocessor and/or a microcontroller.

In block 450, acquire and digitize a plurality of Electroencephalography (EEG) channels from the at least one of the plurality of sub modules for a first Electroencephalography (EEG) data processing and analysis. In one example a plurality of Electroencephalography (EEG) channels from the at least one of the plurality of sub modules is acquired and digitized for a first Electroencephalography (EEG) data processing and analysis.

In one example, the acquisition includes amplification and filtering of the plurality of EEG channels. In one example, the digitization of the plurality of EEG channels is performed by an analog-to-digital converter (ADC). In one example, the first EEG data processing and analysis is performed by a digital signal processor and computer. In one example, the ADC samples its input with a sample clock with a sampling frequency consistent with a signal bandwidth established by the filter. For example, the sampling frequency may be at least a factor of two greater than the signal bandwidth.

In one example, the first EEG data processing and analysis may be over a temporal dimension (i.e., time), a spectral dimension (i.e., frequency) or a spatial dimension (i.e., location) to assist in clinical diagnosis of neurological disorders. In one example, the step of block 450 may be performed (either partially performed or fully performed) by one or more of the following: an electrode (e.g., EEG electrode 110), an amplifier (e.g., EEG signal amplifier 120), a filter (e.g., filter 130), an analog-to-digital converter (ADC) (e.g., ADC 140) and/or an Electroencephalography (EEG) channel signal acquisition equipment as illustrated in FIG. 1.

In block 460, activate one or more of the plurality of sub modules using the staircase shifting with the plurality of source control lines for a second Electroencephalography (EEG) data processing and analysis, wherein the one or more of the plurality of sub modules does not include any of the at least one of the plurality of sub modules. In one example, one or more of the plurality of sub modules is activated using the staircase shifting with the plurality of source control lines for a second Electroencephalography (EEG) data processing and analysis, wherein the one or more of the plurality of sub modules does not include any of the at least one of the plurality of sub modules.

In one example, the reallocation modifies sampling of the spatial dimension of the EEG system. In one example, the step of block 460 may be performed by one or more of the following: a power manager (e.g., a device that manages dc power), a power controller, a microprocessor and/or a microcontroller.

In one aspect, one or more of the steps for providing an EEG system staircase shifting in FIG. 4 may be executed by one or more processors which may include hardware, software, firmware, etc. The one or more processors, for example, may be used to execute software or firmware needed to perform the steps in the flow diagram of FIG. 4. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

The software may reside on a computer-readable medium. The computer-readable medium may be a non-transitory computer-readable medium. A non-transitory computer-readable medium includes, by way of example, a magnetic storage device (e.g., hard disk, floppy disk, magnetic strip), an optical disk (e.g., a compact disc (CD) or a digital versatile disc (DVD)), a smart card, a flash memory device (e.g., a card, a stick, or a key drive), a random access memory (RAM), a read only memory (ROM), a programmable ROM (PROM), an erasable PROM (EPROM), an electrically erasable PROM (EEPROM), a register, a removable disk, and any other suitable medium for storing software and/or instructions that may be accessed and read by a computer. The computer-readable medium may also include, by way of example, a carrier wave, a transmission line, and any other suitable medium for transmitting software and/or instructions that may be accessed and read by a computer. The computer-readable medium may reside in a processing system, external to the processing system, or distributed across multiple entities including the processing system. The computer-readable medium may be embodied in a computer program product. By way of example, a computer program product may include a computer-readable medium in packaging materials. The computer-readable medium may include software or firmware. Those skilled in the art will recognize how best to implement the described functionality presented throughout this disclosure depending on the particular application and the overall design constraints imposed on the overall system.

Any circuitry included in the processor(s) is merely provided as an example, and other means for carrying out the described functions may be included within various aspects of the present disclosure, including but not limited to the instructions stored in the computer-readable medium, or any other suitable apparatus or means described herein, and utilizing, for example, the processes and/or algorithms described herein in relation to the example flow diagram.

Within the present disclosure, the word “exemplary” is used to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation. The term “coupled” is used herein to refer to the direct or indirect coupling between two objects. For example, if object A physically touches object B, and object B touches object C, then objects A and C may still be considered coupled to one another—even if they do not directly physically touch each other. The terms “circuit” and “circuitry” are used broadly, and intended to include both hardware implementations of electrical devices and conductors that, when connected and configured, enable the performance of the functions described in the present disclosure, without limitation as to the type of electronic circuits, as well as software implementations of information and instructions that, when executed by a processor, enable the performance of the functions described in the present disclosure.

One or more of the components, steps, features and/or functions illustrated in the figures may be rearranged and/or combined into a single component, step, feature or function or embodied in several components, steps, or functions. Additional elements, components, steps, and/or functions may also be added without departing from novel features disclosed herein. The apparatus, devices, and/or components illustrated in the figures may be configured to perform one or more of the methods, features, or steps described herein. The novel algorithms described herein may also be efficiently implemented in software and/or embedded in hardware.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

The previous description is provided to enable any person skilled in the art to practice the various aspects described herein. Various modifications to these aspects will be readily apparent to those skilled in the art, and the principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

One skilled in the art would understand that various features of different embodiments may be combined or modified and still be within the spirit and scope of the present disclosure.

What is claimed is:

1. An apparatus comprising:
a plurality of sub modules each configured to acquire an Electroencephalography (EEG) signal;
a main module coupled to the plurality of sub modules, the main module configured to serve as a data orchestrator for a plurality of sub modules:
a plurality of transmission lines coupled to the main module and the plurality of submodules, wherein the plurality of transmission lines is configured to transport a plurality of control lines, and wherein a systematic assignment of the plurality of transmission lines is implemented without external interconnection modification.

2. The apparatus of claim 1, further comprising an Electroencephalography (EEG) electrode coupled to one of the plurality of sub modules, the EEG electrode configured to receive the EEG signal from a brain.

3. The apparatus of claim 2, further comprising an Electroencephalography (EEG) amplifier coupled to the EEG electrode, the EEG amplifier configured to amplify the EEG signal.

4. The apparatus of claim 3, further comprising a filter coupled to the EEG amplifier, the filter configured to restrict the EEG signal to a bandwidth.

5. The apparatus of claim 4, further comprising an analog-to-digital converter (ADC) coupled to the filter, the ADC configured to convert the EEG signal to a digitized Electroencephalography (EEG) signal.

6. The apparatus of claim 1, wherein the main module is further configured to assign one of a plurality of inputs of the plurality of control lines to one of a plurality of outputs of the plurality of control lines to implement a unique control line to each of the plurality of sub modules.

7. A method comprising:
establishing a first control plane communication between a main module and a first of a plurality of sub modules with a plurality of source control lines;
establishing a second control plane communication between the first and a second of the plurality of sub modules with the plurality of source control lines using a staircase shifting; and
activating at least one of the plurality of sub modules using the staircase shifting with the plurality of source control lines.

8. The method of claim 7, wherein the at least one of the plurality of sub modules includes the first of the plurality of sub modules or the second of the plurality of sub modules.

9. The method of claim 7, further comprising designating one of the plurality of source control lines for communicating with one of the plurality of sub modules.

10. The method of claim 9, further comprising assigning one of a plurality of inputs of the plurality of source control lines to one of a plurality of outputs of the plurality of source control lines to implement a unique source control line to each of the plurality of sub modules.

11. The method of claim 10, further comprising acquiring and digitizing a plurality of Electroencephalography (EEG) channels from the at least one of the plurality of sub modules for a first Electroencephalography (EEG) data processing and analysis.

12. The method of claim 11, further comprising establishing a data plane communication between the main module and the plurality of sub modules in an Electroencephalography (EEG) system with an outbound serial data interface and an inbound serial data interface.

13. The method of claim 12, further comprising activating one or more of the plurality of sub modules using the staircase shifting with the plurality of source control lines for a second Electroencephalography (EEG) data processing and analysis, wherein the one or more of the plurality of sub modules does not include any of the at least one of the plurality of sub modules.

14. A non-transitory computer-readable medium storing computer executable code, operable on a device comprising at least one processor and at least one memory coupled to the at least one processor, wherein the at least one processor is configured to implement Electroencephalography (EEG) scalable channel capacity, the computer executable code comprising:
instructions for causing a computer to establish a first control plane communication between a main module and a first of a plurality of sub modules with a plurality of source control lines;
instructions for causing the computer to establish a second control plane communication between the first and a second of the plurality of sub modules with the plurality of source control lines using a staircase shifting; and
instructions for causing the computer to activate at least one of the plurality of sub modules using the staircase shifting with the plurality of source control lines.

15. The non-transitory computer-readable medium of claim 14, wherein the at least one of the plurality of sub modules includes the first of the plurality of sub modules or the second of the plurality of sub modules.

16. The non-transitory computer-readable medium of claim 14, further comprising instructions for causing the computer to designate one of the plurality of source control lines for communicating with one of the plurality of sub modules.

17. The non-transitory computer-readable medium of claim 14, further comprising instructions for causing the computer to assign one of a plurality of inputs of the plurality of source control lines to one of a plurality of outputs of the plurality of source control lines to implement a unique source control line to each of the plurality of sub modules.

18. The non-transitory computer-readable medium of claim 17, further comprising instructions for causing the computer to acquire and digitize a plurality of Electroencephalography (EEG) channels from the at least one of the plurality of sub modules for a first Electroencephalography (EEG) data processing and analysis.

19. The non-transitory computer-readable medium of claim 18, further comprising instructions for causing the computer to establish a data plane communication between the main module and the plurality of sub modules in an Electroencephalography (EEG) system with an outbound serial data interface and an inbound serial data interface.

20. The non-transitory computer-readable medium of claim 19, further comprising instructions for causing the computer to activate one or more of the plurality of sub modules using the staircase shifting with the plurality of source control lines for a second Electroencephalography (EEG) data processing and analysis, wherein the one or more of the plurality of sub modules does not include any of the at least one of the plurality of sub modules.
